(19) United States
(12) Patent Application Publication
(10) Pub. No.: US 2009/0105823 A1
(43) Pub. Date: Apr. 23, 2009

(54) Artificial intervertebral disc nucleus

(75) Inventors: Michael S. Williams, Santa Rosa, CA (US); Jeffrey A. Smith, Petaluma, CA (US)

Correspondence Address: Deanna J Shirley, 3418 Baldwin way, Santa Rosa, CA, 95403, US

(73) Assignee: SYNECOR, LLC, DURHAM, NORTH CAROLINA, NC (US)

(21) Appl. No.: 11/659,437

(22) PCT Filed: Sep. 30, 2005
(86) PCT No.: PCT/US05/35035

§ 371 Date: Oct. 30, 2008

Related U.S. Application Data
(60) Provisional application No. 60/614,702, filed on Sep. 30, 2004.

(51) Int. Cl. A61F 2/44

(52) U.S. Cl. 623/17.16; 623/17.11

(57) ABSTRACT

Devices and methods for manufacturing devices for treating degenerated and/or traumatized intervertebral discs are disclosed. Artificial discs and components of discs may include an artificial nucleus and/or an artificial annulus and may be comprised of shape memory materials synthesized to achieve desired mechanical and physical properties. An artificial nucleus and/or annulus according to the invention may comprise a filler retention membrane that may be filled with a curable material for deployment. A filler retention membrane according to the invention may comprise one or more partitions to define one or more chambers and may comprise one or more valves for selectively permitting the flow of material within the nuclear region and annular region of the filler retention membrane.

### RELATED APPLICATIONS

This application is related to and claims the benefit of the priority date of U.S. Provisional Patent Application Ser. No. 60/614,702 entitled “Artificial Intervertebral Disc Nucleus”, filed Sep. 30, 2004 by Smith, et al.; U.S. patent application Ser. No. 10/990,158, entitled “Artificial Intervertebral Disc”, filed Nov. 16, 2004 by Williams, et al.

### FIELD OF THE INVENTION

The invention herein relates generally to medical devices and methods of treatment, and more particularly to devices and methods used in the treatment of a degenerated intervertebral disc and/or disc nucleus.

### BACKGROUND OF THE INVENTION

Intervertebral disc degeneration is a leading cause of pain and disability, occurring in a substantial majority of people at some point during adulthood. The intervertebral disc, comprising primarily the nucleus pulposus and surrounding annulus fibrosus, constitutes a vital component of the functional spinal unit. The intervertebral disc maintains space between adjacent vertebral bodies, absorbs impact between and cushions the vertebral bodies. The disc allows for fluid movement between the vertebral bodies, both subtle (for example, with each breath inhaled and exhaled) and dramatic (including rotational movement and bending movement in all planes.) Deterioration of the biological and mechanical integrity of an intervertebral disc as a result of disease and/or aging may limit mobility and produce pain, either directly or indirectly as a result of disruption of the functioning of the spine. Estimated health care costs of treating disc degeneration in the United States exceed $60 billion annually.

Age-related disc changes are progressive, and, once significant, increase the risk of related disorders of the spine. The degenerative process alters intradiscal pressures, causing a relative shift of axial load-bearing to the peripheral regions of the endplates and facets of the vertebral bodies. Such a shift promotes abnormal loading of adjacent intervertebral discs and vertebral bodies, altering spinal balance, shifting the axis of rotation of the vertebral bodies, and increasing risk of injury to these units of the spine. Further, the transfer of biomechanical loads appears to be associated with the development of other disorders, including both facet and ligament hypertrophy, osteophyte formation, lyphosis, spondylolisthesis, nerve damage, and pain.

In addition to age-related changes, numerous individuals suffer trauma-induced damage to the spine including the intervertebral discs. Trauma induced damage may include ruptures, tears, prolapse, herniations, and other injuries that cause pain and reduce strength and function.

Non-operative therapeutic options for individuals with neck and back pain include rest, analgesics, physical therapy, heat, and manipulation. These treatments fail in a significant number of patients. Current surgical options for spinal disease include discectomy, discectomy combined with fusion, and fusion alone. Numerous discectomies are performed annually in the United States. The procedure is effective in promptly relieving significant radicular pain, but, in general, the return of pain increases proportionally with the length of time following surgery. In fact, the majority of patients experience significant back pain by ten years following lumbar discectomy.

An attempt to overcome some of the possible reasons for failure of discectomy, fusion has the potential to maintain normal disc space height, to eliminate spine segment instability, and eliminate pain by preventing motion across a destabilized or degenerated spinal segment.

However, although some positive results are possible, spinal fusion may have harmful consequences as well. Fusion involves joining portions of adjacent vertebrae to one another. Because motion is eliminated at the treated level, the biomechanics of adjacent levels are disrupted. Resulting pathological processes such as spinal stenosis, disc degeneration, osteophyte formation, and others may occur at levels adjacent to a fusion, and cause pain in many patients. In addition, depending upon the device or devices and techniques used, surgery may be invasive and require a lengthy recovery period.

Consequently, there is a need in the art to treat degenerative disc disease and/or traumatized intervertebral discs, while eliminating the shortcomings of the prior art. There remains a need in the art to achieve the benefit of removal of a non-functioning intervertebral disc, to replace all or a portion of the disc with a device that will function as a healthy disc, eliminating pain, while preserving motion. There remains a need for an artificial disc or other device that maintains the proper intervertebral spacing, allows for motion, distributes axial load appropriately, and provides stability. In addition, an artificial disc requires secure long-term fixation to bone.

Further, there remains a need for an artificial nucleus that can be implanted within the annulus fibrosus, in order to restore normal disc functioning Such a nucleus must comprise the characteristic lower durometer than the annulus fibrosus, and the annulus fibrosus must comprise the requisite stiffness as compared with the nucleus. Further, there remains a need for an artificial disc that can withstand typical cyclic stresses and perform throughout the life a patient. An artificial disc that can be implanted using minimally invasive techniques is also needed. And finally, a device that is compatible with current imaging modalities, such as Magnetic Resonance Imaging (MRI) is needed.

### SUMMARY OF THE INVENTION

An endoprosthesis for partial or complete replacement of an intervertebral disc is disclosed comprising an inner nuclear region and an outer nuclear region, wherein the inner nuclear region is defined by one or more walls, and wherein one or more of the walls comprises one or more valves. The endoprosthesis may further comprise a flowable filler medium, wherein the walls substantially prevent the flow of the filler medium between the inner and outer nuclear regions, and a valve or valves selectively permit the flow of the medium between the inner and outer nuclear regions. Further, the outer nuclear region and/or the inner nuclear region may comprise one or more partitions which in turn may comprise one or more valves which permit the flow of material within the region. The device may comprise means for measuring pressure within at least one of said inner nuclear region and said outer nuclear region.

An embodiment according to the invention may comprise a filler retention membrane comprising an interior, a neck and an orifice, wherein the neck and the orifice are disposed substantially within the interior of the filler retention membrane. The orifice may comprise one or more sealed regions which may be disposed laterally with respect to the orifice either contiguously with the orifice, with the exterior walls, or both. The neck may further be anchored within the interior of said filler retention membrane. The interior of the neck may comprise a substantially solid material.

An endoprosthesis for partial or complete replacement of an intervertebral disc according to the invention may comprise an exterior and an interior, an exterior port and an interior port and a passage therebetween, wherein the exterior port is offset with respect to the interior port. The passage may comprise a non-linear configuration, such as, for example, an ‘S’ shaped or ‘Z’ shaped configuration. The passage may also comprise a substantially solid material.

An alternative embodiment according to the invention may comprise a principle nuclear region and a reinforcement region, wherein the cross-sectional configuration of the reinforcement region is of a polygonal configuration. The reinforcement region may be disposed substantially within the interior of said principle nuclear portion or substantially about the exterior of the principle nuclear region.

Yet another endoprosthesis for partial or complete replacement of an intervertebral disc may comprise a primary filler retention membrane and one or more secondary filler retention membranes disposed within the primary filler retention membrane. The primary and said secondary filler retention membranes comprise a delivery configuration and a deployed configuration, and may further comprise one or more means for measuring pressure within the interior of the primary or secondary filler retention membranes, or both.

### BRIEF DESCRIPTION OF THE DRAWINGS

FIG. 1 is a generally central cross-sectional plan view of an embodiment according to the invention in its deployed configuration at equilibrium.

FIG. 2 is the cross-sectional plan view of the embodiment of FIG. 1 after being subjected to a force.

FIG. 3 illustrates a generally central cross-sectional side view of a balloon following conventional manufacture.

FIG. 4 is the cross-sectional side view of the balloon of FIG. 3 following a step of manufacture according to the invention.

FIG. 5 is a cross-sectional end view of a portion of the balloon of FIG. 4 prepared according to the invention.

FIG. 6 is a generally central cross-sectional side view of an embodiment according to the invention.

FIG. 7 is a generally central cross-sectional plan view of yet another embodiment according to the invention.

FIG. 8 is a cross-sectional side view of yet another embodiment according to the invention.

FIG. 9 is a cross-sectional side view of yet another embodiment according to the invention.

FIG. 10 is a cross-sectional view of yet another embodiment according to the invention.

FIG. 11 is a cross-sectional view of yet another embodiment according to the invention.

### DETAILED DESCRIPTION OF THE INVENTION

An endoprosthesis known as an artificial disc and/or an artificial disc nucleus are designed to replace a degenerated intervertebral disc. Such an artificial disc or disc nucleus may be expandable and/or self-expanding.

An “expandable” endoprosthesis comprises a reduced profile configuration and an expanded profile configuration. An expandable endoprosthesis according to the invention may undergo a transition from a reduced configuration to an expanded profile configuration via any suitable means, or may be self-expanding. Some embodiments according to the invention may comprise a substantially hollow interior that may be filled with a suitable medium, examples of which are set forth below. Such embodiments may accordingly be introduced into the body in a collapsed configuration, and, following introduction, may be filled to form a deployed configuration. Embodiments according to the invention may accordingly be implanted percutaneously or surgically. If implanted surgically, embodiments according to the invention may be implanted from either an anterior or a posterior approach, following the removal of some or all of the native disc, excepting the periphery of the native nucleus.

“Spinal fusion” is a process by which one or more adjacent vertebral bodies are adjoined to one another in order to eliminate motion across an unstable or degenerated spinal segment.

“Preservation of mobility” refers to the desired maintenance of normal motion between separate spinal segments.

“Spinal unit” refers to a set of the vital functional parts of the spine including a vertebral body, endplates, facets, and intervertebral disc.

The term “cable” refers to any generally elongate member fabricated from any suitable material, whether polymeric, metal or metal alloy, natural or synthetic.

The term “fiber” refers to any generally elongate member fabricated from any suitable material, whether polymeric, metal or metal alloy, natural or synthetic.

As used herein, the term “braid” refers to any braid or mesh or similar wound or woven structure produced from between 1 and several hundred longitudinal and/or transverse elongate elements wound, woven, braided, knitted, helically wound, or intertwined by any manner, at angles between 0 and 180 degrees and usually between 45 and 105 degrees, depending upon the overall geometry and dimensions desired.

Unless specified, suitable means of attachment may include by thermal melt, chemical bond, adhesive, sintering, welding, or any means known in the art.

As used herein, a device is “implanted” if it is placed within the body to remain for any length of time following the conclusion of the procedure to place the device within the body.

The term “diffusion coefficient” refers to the rate by which a substance elutes, or is released either passively or actively from a substrate.

Unless specified, suitable means of attachment may include by thermal melt, chemical bond, adhesive, sintering, welding, or any means known in the art.

“Shape memory” refers to the ability of a material to undergo structural phase transformation such that the material may define a first configuration under particular physical and/or chemical conditions, and to revert to an alternate configuration upon a change in those conditions. Shape memory materials may be metal alloys including but not limited to nickel titanium, or may be polymeric. A polymer is a shape memory polymer if the original shape of the polymer is recovered by heating it above a shape recovering temperature (defined as the transition temperature of a soft segment) even if the original molded shape of the polymer is destroyed mechanically at a lower temperature than the shape recovering temperature, or if the memorized shape is recoverable by application of another stimulus. Such other stimulus may include but is not limited to pH, salinity, hydration, radiation, including but not limited to radiation in the ultraviolet range, and others. Some embodiments according to the invention may comprise one or more polymers having a structure that assumes a first configuration, a second configuration, and a hydrophilic polymer of sufficient rigidity coated upon at least a portion of the structure when the device is in the second configuration. Upon placement of the device in an aqueous environment and consequent hydration of the hydrophilic polymer, the polymer structure reverts to the first configuration.

Some embodiments according to the invention, while not technically comprising shape memory characteristics, may nonetheless readily convert from a constrained configuration to a deployed configuration upon removal of constraints, as a result of a material's elasticity, super-elasticity, a particular method of “rolling down” and constraining the device for delivery, or a combination of the foregoing. Such embodiments may comprise one or more elastomeric or rubber materials.

As used herein, the term “segment” refers to a block or sequence of polymer forming part of the shape memory polymer. The terms hard segment and soft segment are relative terms, relating to the transition temperature of the segments. Generally speaking, hard segments have a higher glass transition temperature than soft segments, but there are exceptions.

“Transition temperature” refers to the temperature above which a shape memory polymer reverts to its original memorized configuration.

The term “strain fixity rate” R_{f} is a quantification of the fixability of a shape memory polymer's temporary form, and is determined using both strain and thermal programs. The strain fixity rate is determined by gathering data from heating a sample above its melting point, expanding the sample to 200% of its temporary size, cooling it in the expanded state, and drawing back the extension to 0%, and employing the mathematical formula:

R_{f}(N)=εₙ(N)/εₘ

where εᵤ(N) is the extension in the tension-free state while drawing back the extension, and εₘ, is 200%.
The “strain recovery rate” Rᵣ describes the extent to which the permanent shape is recovered:

$${R}_{r} ( N ) = \frac{{ɛ}_{m} - {ɛ}_{p} ( N )}{{ɛ}_{m} - {ɛ}_{p} ( N - 1 )}$$

where εₚ is the extension at the tension free state.

A “switching segment” comprises a transition temperature and is responsible for the shape memory polymer's ability to fix a temporary shape.

A “thermoplastic elastomer” is a shape memory polymer comprising crosslinks that are predominantly physical crosslinks.

A “thermoset” is a shape memory polymer comprising a large number of crosslinks that are covalent bonds.

Shape memory polymers are highly versatile, and many of the advantageous properties listed above are readily controlled and modified through a variety of techniques. Several macroscopic properties such as transition temperature and mechanical properties can be varied in a wide range by only small changes in their chemical structure and composition. More specific examples are set forth in Provisional U.S. Patent Application Ser. No. 60/523,578 and are incorporated in their entirety as if fully set forth herein.

Shape memory polymers are characterized by two features, triggering segments having a thermal transition Tₜᵣₐₙₛ within the temperature range of interest, and crosslinks determining the permanent shape. Depending on the kind of crosslinks (physical versus covalent bonds), shape memory polymers can be thermoplastic elastomers or thermosets. By manipulating the types of crosslinks, the transition temperature, and other characteristics, shape memory polymers can be tailored for specific clinical applications.

More specifically, according the invention herein, one can the control shape memory behavior and mechanical properties of a shape memory polymer through selection of segments chosen for their transition temperature, and mechanical properties can be influenced by the content of respective segments. The extent of crosslinking can be controlled depending on the type of material desired through selection of materials where greater crosslinking makes for a tougher material than a polymer network. In addition, the molecular weight of a macromonomeric crosslinker is one parameter on the molecular level to adjust crystallinity and mechanical properties of the polymer networks. An additional monomer may be introduced to represent a second parameter.

Further, the annealing process (comprising heating of the materials according to chosen parameters including but not limited to time and temperature) increases polymer chain crystallization, thereby increasing the strength of the material. Consequently, according to the invention, the desired material properties can be achieved by using the appropriate ratio of materials and by annealing the materials.

Additionally, the properties of polymers can be enhanced and differentiated by controlling the degree to which the material crystallizes through strain-induced crystallization. Means for imparting strain-induced crystallization are enhanced during deployment of an endoprosthesis according to the invention. Upon expansion of an endoprosthesis according to the invention, focal regions of plastic deformation undergo strain-induced crystallization, further enhancing the desired mechanical properties of the device, such as further increasing radial strength. The strength is optimized when the endoprosthesis is induced to bend preferentially at desired points.

Natural polymer segments or polymers include but are not limited to proteins such as casein, gelatin, gluten, zein, modified zein, serum albumin, and collagen, and polysaccharides such as alginate, chitin, celluloses, dextrans, pullulane, and polyhyaluronic acid; poly(3-hydroxyalkanoate)s, especially poly(.beta.-hydroxybutyrate), poly(3-hydroxyoctanoate) and poly(3-hydroxyfatty acids).

Suitable synthetic polymer blocks include polyphosphazenes, poly(vinyl alcohols), polyamides, polyester amides, poly(amino acid)s, synthetic poly(amino acids), polycarbonates, polyacrylates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyesters, polyethylene terephthalate, polysiloxanes, polyurethanes, fluoropolymers (including but not limited to polyfluorotetraethylene), and copolymers thereof.

Examples of suitable polyacrylates include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate) and poly(octadecyl acrylate).

Synthetically modified natural polymers include cellulose derivatives such as alkyl celluloses, hydroxyalkyl celluloses, cellulose ethers, cellulose esters, nitrocelluloses, and chitosan. Examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, arboxymethyl cellulose, cellulose triacetate and cellulose sulfate sodium salt. These are collectively referred to herein as “celluloses”.

For those embodiments comprising a shape memory polymer, the degree of crystallinity of the polymer or polymeric block(s) is between 3 and 80%, more often between 3 and 65%. The tensile modulus of the polymers below the transition temperature is typically between 50 MPa and 2 GPa (gigapascals), whereas the tensile modulus of the polymers above the transition temperature is typically between 1 and 500 MPa. Most often, the ratio of elastic modulus above and below the transition temperature is 20 or more.

The melting point and glass transition temperature of the hard segment are generally at least 10 degrees C., and preferably 20 degrees C., higher than the transition temperature of the soft segment. The transition temperature of the hard segment is preferably between −60 and 270 degrees C., and more often between 30 and 150 degrees C. The ratio by weight of the hard segment to soft segments is between about 5:95 and 95:5, and most often between 20:80 and 80:20. The shape memory polymers contain at least one physical crosslink (physical interaction of the hard segment) or contain covalent crosslinks instead of a hard segment. The shape memory polymers can also be interpenetrating networks or semi-interpenetrating networks. A typical shape memory polymer is a block copolymer.

Examples of suitable hydrophilic polymers include but are not limited to poly(ethylene oxide), polyvinyl pyrrolidone, polyvinyl alcohol, poly(ethylene glycol), polyacrylamide poly(hydroxy alkyl methacrylates), poly(hydroxy ethyl methacrylate), hydrophilic polyurethanes, HYPAN, oriented HYPAN, poly(hydroxy ethyl acrylate), hydroxy ethyl cellulose, hydroxy propyl cellulose, methoxylated pectin gels, agar, starches, modified starches, alginates, hydroxy ethyl carbohydrates and mixtures and copolymers thereof.

Hydrogels can be formed from polyethylene glycol, polyethylene oxide, polyvinyl alcohol, polyvinyl pyrrolidone, polyacrylates, poly (ethylene terephthalate), poly(vinyl acetate), and copolymers and blends thereof. Several polymeric segments, for example, acrylic acid, are elastomeric only when the polymer is hydrated and hydrogels are formed. Other polymeric segments, for example, methacrylic acid, are crystalline and capable of melting even when the polymers are not hydrated. Either type of polymeric block can be used, depending on the desired application and conditions of use.

Examples of highly elastic materials including but not limited to vulcanized rubber, polyurethanes, thermoplastic elastomers, and others may be used according to the invention.

Curable materials include any material capable of being able to transform from a fluent or soft material to a harder material, by cross-linking, polymerization, or other suitable process. Materials may be cured over time, thermally, chemically, or by exposure to radiation. For those materials that are cured by exposure to radiation, many types of radiation may be used, depending upon the material. Wavelengths in the spectral range of about 100-1300 nm may be used. The material should absorb light within a wavelength range that is not readily absorbed by tissue, blood elements, physiological fluids, or water. Ultraviolet radiation having a wavelength ranging from about 100-400 nm may be used, as well as visible, infrared and thermal radiation. The following materials are some examples of curable materials: urethanes, polyurethane oligomer mixtures, acrylate monomers, aliphatic urethane acrylate oligomers, acrylamides, UV curable epoxies, photopolymerizable polyanhydrides and other UV curable monomers. Alternatively, the curable material can be a material capable of being chemically cured, such as silicone based compounds which undergo room temperature vulcanization.

Though not limited thereto, some embodiments according to the invention comprise one or more therapeutic substances within a filler medium or that will elute from the surface. Suitable therapeutics include but are not limited to bone growth accelerators, bone growth inducing factors, osteoinductive agents, immunosuppressive agents, steroids, anti-inflammatory agents, pain management agents (e.g, analgesics), tissue proliferative agents to enhance regrowth and/or strengthening of native disc materials, agents to promote angiogenesis, agents to inhibit calcification, and others. According to the invention, such surface treatment and/or incorporation of therapeutic substances may be performed utilizing one or more of numerous processes that utilize carbon dioxide fluid, e.g., carbon dioxide in a liquid or supercritical state. A supercritical fluid is a substance above its critical temperature and critical pressure (or “critical point”).

The use of polymeric materials in the fabrication of endoprostheses confers the advantages of improved flexibility, compliance and conformability. Fabrication of an endoprosthesis according to the invention allows for the use of different materials in different regions of the prosthesis to achieve different physical properties as desired for a selected region. An endoprosthesis comprising polymeric materials has the additional advantage of compatibility with magnetic resonance imaging, potentially a long-term clinical benefit.

As set forth above, some embodiments according to the invention may comprise components that have a substantially hollow interior that may be filled after being delivered to a treatment site with a suitable material in order to place the device in a deployed configuration. Accordingly, such embodiments may comprise a filler retention membrane having a layer comprising polyvinyl chloride (PVC), polyurethane, and or laminates of polyethylene terephthalate (PET) or nylon fibers or films within layers of PVC, polycarbonate polyurethane or other suitable material. Such a filler retention membrane layer alternatively may comprise Kevlar, polyimide, a suitable metal, or other suitable material within layers of PVC, polyurethane or other suitable material. Such laminates may be of solid core, braided, woven, wound, or other fiber mesh structure, and provide stability, strength, and a controlled degree of compliance. Such a laminate membrane layer may be manufactured using radiofrequency or ultrasonic welding, adhesives including ultraviolet curable adhesives, or thermal energy.

A filler retention membrane as set forth above may be filled with any suitable material including but not limited to saline, silicones, polyurethane, contrast media, hydrogels, a polymeric foam, or any combination thereof. A polymeric foam may comprise a polyurethane intermediate comprising polymeric diisocyanate, polyols, and a hydrocarbon, or a carbon dioxide gas mixture. Filler material may be loaded with any of numerous solid or liquid materials known in the art that confer radiopacity.

Such a filler retention membrane may be designed to replace the nucleus pulposus of an intervertebral disc Alternatively, it may replace the entire intervertebral disc or only the annulus fibrosus. Such a device may comprise one or more filling ports, and include separate filling ports for the nucleus pulposus and annulus fibrosus, to allow for varying durometers, and varied materials in order to mimic the properties of the native disc components.

Such a device may comprise a single unit, or may be two or more individual parts. If the device comprises two or more component parts, the parts may fit together in a puzzle-like fashion. The device may further comprise alignment tabs for stable alignment tabs for stable alignment between the vertebral bodies.

Such a filler retention membrane may comprise interbody connections and/or baffles and/or partitions or generally vertically oriented membrane walls in order to maintain structural integrity after filling, to increase the devices ability to withstand compressive, shear, and other loading forces, and/or to direct filling material flow and positioning, and/or to partition portions of the disc in order to separate injection of different types or amounts of filling materials. Such partitions may comprise valves. Further, such a device may comprise a characteristic durometer selected for suitability to the level of the vertebra within the spine for which the intervertebral disc is being treated. For example, an artificial intervertebral disc nucleus within the cervical spine may comprise a lower durometer than a replacement nucleus in the lumbar region.

Following surgical or minimally invasive surgical access and removal of all or a portion of the native disc, a deflated filler retention membrane may be delivered to the intervertebral space surgically or through a catheter and/or cannula. The membrane is positioned within the intervertebral space. The membrane inflation port or ports are then attached to the injection source. Filling material is then injected. Following injection of the filling material, which may be curable by any suitable means, for example, photochemical, chemical, or other means, or may be catalytically activated or may remain in fluid form, the injection source is detached and removed.

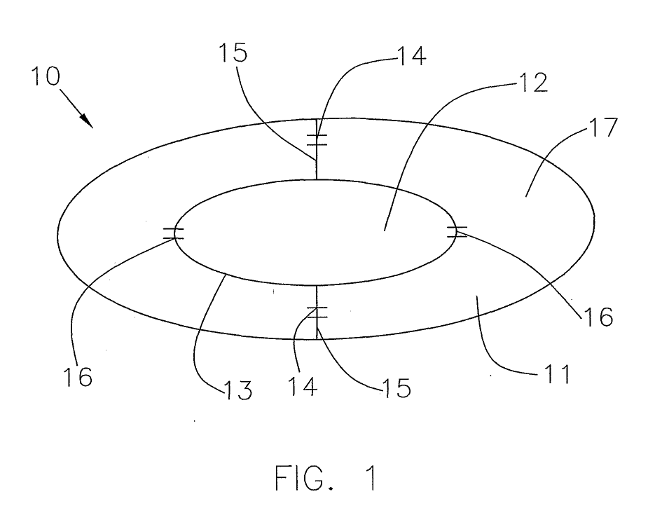

Details of the invention can be better understood from the following descriptions of specific embodiments according to the invention. FIG. 1 illustrates artificial nucleus 10 according to the invention The device may alternatively be a complete artificial disc according to the invention. Here, artificial nucleus 10 comprises substantially hollow outer nucleus region 11 and substantially hollow inner nuclear region 12. Outer nucleus region 11 and inner nucleus region 12 may be filled with substantially fluid medium 17. (Substantially fluid medium may or may not cure or be deployed to form a substantially solid material. Further, substantially fluid medium may comprise a shape memory or other convertible component) Inner nuclear wall 13 separates outer nucleus region 11 from inner nuclear region 12. Partitions 15 divide outer nuclear portion 11. (Alternatively, or in addition, inner nuclear region 12 may comprise one or more partitions.)

Partitions 15 comprise one or more valves 14 and inner nuclear wall 13 comprises one or more valves 16. The exterior wall enclosing outer nuclear region 11 may also comprise one or more ports and one or more seals (not pictured). Fluid medium 17, shown in FIG. 1, is substantially at equilibrium between outer nuclear region 11 and inner nuclear region 12, may pass through valves 14 and valves 16 into inner nuclear region 12. Numerous other configurations of outer nuclear region 11, inner nuclear region 12, wall 13, valves 14 and 16, and partitions 15 are possible and within the scope of the invention. Further, either or both inner nuclear region 12 and outer nuclear region 11 may comprise means for detecting and/or indicating pressure within the interior or either inner nuclear region 12 or outer nuclear region 11. Means for detecting and/or indicating pressure may, for example, be affixed to exterior wall or, for example one or more partitions.

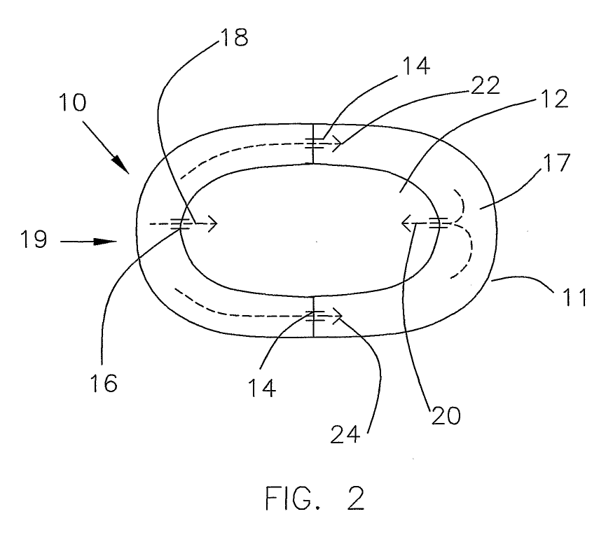

FIG. 2 illustrates artificial disc nucleus 10 of FIG. 1 subjected to lateral force 19. Lateral force 19 initiates flow of substantially fluid medium 17 in the directions of arrows 18, 20, 22 and 24. Fluid medium 17 passes through valves 14 to travel through outer nuclear region 11, and through valves 16 into the inner nuclear region 12. The resulting fluid flow and increased volume of fluid medium 17 within inner nuclear region 12 serve to absorb the impact of force 19. Alternatively, a force (such as, for example, a bidirectional force) exerted upon artificial disc nucleus 10 may result in an alternative direction of flow of material. Further, in an artificial disc nucleus 10 comprising an alternative configuration of partitions and/or valves, an alternative direction of flow of material may result as the nucleus absorbs the impact of the force.

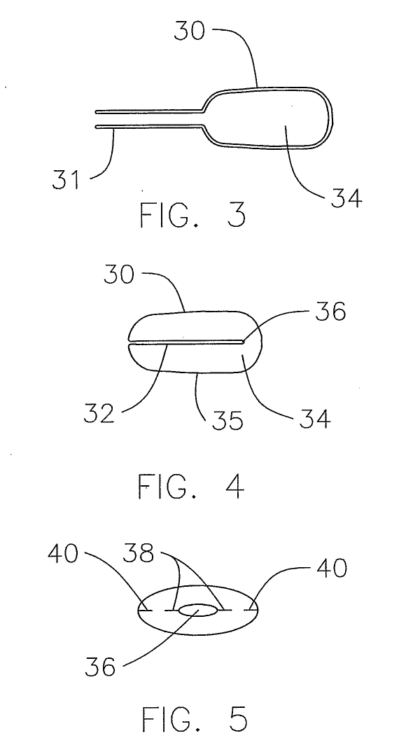

FIGS. 3-5 illustrate sequential steps in the manufacture of a fillable membrane or balloon according to the invention. FIG. 3 depicts a conventional membrane or balloon 30 comprising neck 32 and substantially hollow interior 34. Balloon 30 may be formed from two or more sheets of thin membrane sealed together. Neck 32 of balloon 30 is then sealed according to the illustration of FIG. 5, discussed below. FIG. 4 illustrates balloon 35 according to the invention prepared by inversion of now sealed neck 32 into substantially hollow interior 34. A substantially fluid filler material (not pictured) may be introduced into balloon 35 and/or only neck 32. Such a material may be cured to form a substantially solid material, and consequently, or, in the alternative, a substantially solid material may fill neck 32. A needle may then be used to penetrate material within neck 32 (not pictured) in order to introduce a filler material into balloon 35.

FIG. 5 illustrates an end view of sealed neck 32. Sealed neck 32 comprises opening 36 flanked by neck seals 38. Neck 32 further comprises outer seals 40. The structure and generally elastic material comprising sealed neck 32 and opening 36 prevent the escape of filler material, and opening 36 maintains a “flattened” configuration after filling of balloon 30. The membrane may be sealed by, for example, laser, adhesive, heat, or other process known in the art.

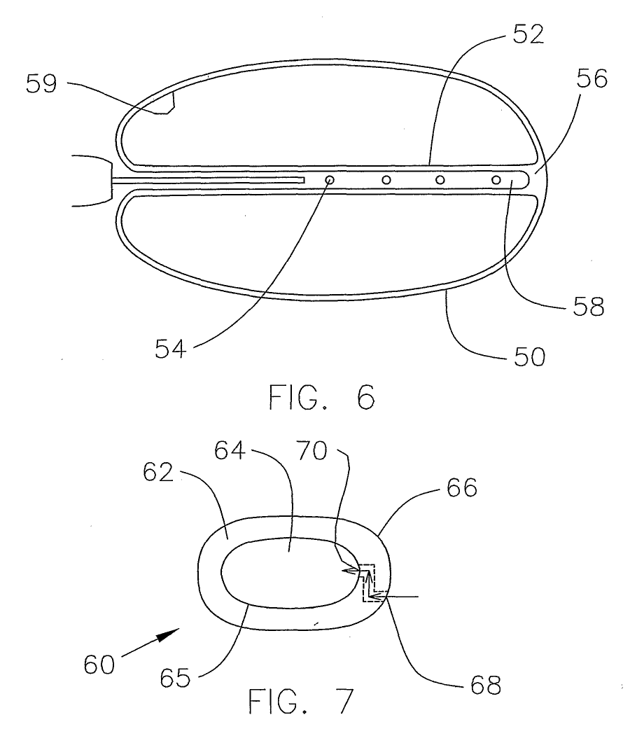

FIG. 6 illustrates an alternative embodiment of the invention Balloon 50 is similar to balloon 30 of FIG. 4. Balloon 50 comprises inverted sealed neck 52 comprising fill ports 54. However, sealed neck 52 further comprises end seal 56, which anchors end 58 of neck 52 to the interior wall 59 of balloon 50.

Turning now to FIG. 7, an alternative embodiment according to the invention is illustrated. FIG. 7 is a cross-sectional view of artificial disc nucleus 60, comprising outer region 62 and inner region 64, which may be of varying durometer and/or filled with materials which are of or cure to form materials of varying durometer. Exterior wall 66 of artificial disc nucleus 60 comprises port 68. Port 68 traverses exterior wall 66 in a generally zig-zag path before breaching inner region wall 65 at interior port 70. Consequently, port 68 does not directly communicate with interior port 70, thereby preventing escape of any substantially fluid filler material (not shown) introduced into inner region 64 via port 68. Port 68 could alternatively travel, for example, an “S” shaped, or other indirect or irregular path according to the invention. In some embodiments, a filler material (not shown) may cure to a relatively solid material

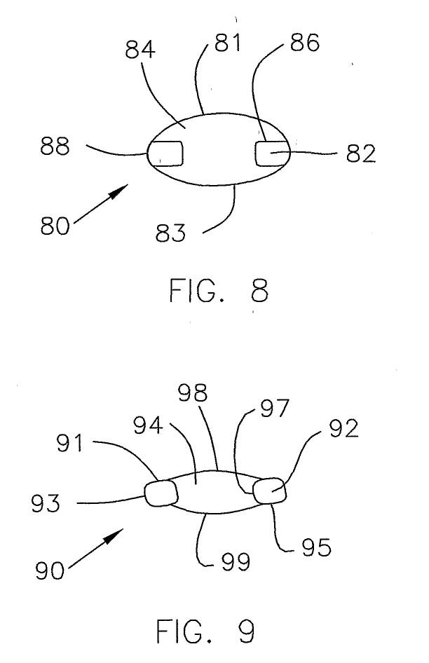

FIG. 8 illustrates a cross-section of artificial disc nucleus 80. Disc nucleus 80 comprises reinforcing region 82 and principle nuclear region 84. As illustrated, peripheral wall or walls 86 of reinforcing region 82 comprise a generally square or rectangular cross section. Peripheral wall or walls 86 may alternatively comprise an ovular, circular, or elliptical cross section. Reinforcing region 82 is generally interior to nucleus 80, with the exception of outer wall 88. The cross section of principle nuclear region 84 may similarly be of alternative configuration, and may include generally centralized convex portions (not pictured) at its superior and inferior surfaces 81 and 83 respectively.

Differing somewhat from the embodiment of FIG. 8, artificial nucleus 90, illustrated in FIG. 9, comprises reinforcing region 92 and principle nuclear region 94. Annular walls 96 comprise a generally square of rectangular cross section. Peripheral walls 97 of reinforcing region 92 may also alternatively comprise an ovular, circular, or elliptical cross section. Superior surface 91, lateral surface 93, and inferior surface 95 of reinforcing region 92 are all exposed as the exterior of disc nucleus 90. The cross section of principle nuclear region 94 may similarly be of alternative configuration, and may include generally centralized convex portions (not pictured) at its superior and inferior surfaces 98 and 99 respectively.

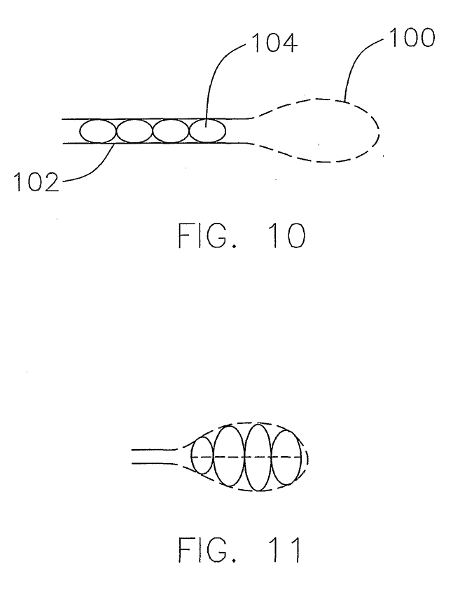

FIG. 10 and FIG. 11 illustrate an alternative embodiment according to the invention at various stages of deployment. FIG. 10 illustrates balloon 100 comprising neck 102 through which one or more fillable membranes 104 are being inserted. Fillable membranes 104, in their low-profile delivery configuration, are percutaneously delivered substantially near distal end 106 of balloon 100. Following delivery, fillable membranes 104 are deployed, either by the introduction of or the deployment of a filling material (not pictured) within the interior of fillable membrane or membranes 104. FIG. 11 illustrates an example of such fillable membranes following deployment. Following deployment, fillable membranes 104 comprise a larger volume, larger profile deployed configuration. Filling material may comprise a substantially fluid material, and/or may be cured or exposed to a stimulus in order to transform to an alternative state, a larger volume, and/or a more solid state.

While all of the foregoing embodiments can most advantageously be delivered in a minimally invasive, percutaneous manner, the foregoing embodiments may also be implanted surgically. Further, while particular forms of the invention have been illustrated and described above, the foregoing descriptions are intended as examples, and to one skilled in the art it will be apparent that various modifications can be made without departing from the spirit and scope of the invention.

We claim:

1. An endoprosthesis for partial or complete replacement of an intervertebral disc, said endoprosthesis comprising an inner nuclear region and an outer nuclear region, wherein said inner nuclear region is defined by one or more walls, and wherein one or more of said walls comprises one or more valves

2. The endoprosthesis of claim 1 further comprising a flowable filler medium, wherein said one or more walls substantially prevents the flow of said filler medium between said inner nuclear region and said outer nuclear region, and wherein said one or more valves selectively permits the flow of said filler medium between said inner nuclear region and said outer nuclear region.

3. The endoprosthesis of claim 1 wherein said outer nuclear region comprises one or more partitions and wherein one or more of said partitions comprises one or more valves.

4. The endoprosthesis of claim 3 further comprising a flowable filler medium, wherein said one or more partitions substantially prevents the flow of said filler medium within said outer nuclear region, and wherein said one or more valves selectively permits the flow of said filler medium within said outer nuclear region.

5. The endoprosthesis of claim 1 wherein said endoprosthesis further comprises means for measuring pressure within at least one of said inner nuclear region and said outer nuclear region.

6. An endoprosthesis for partial or complete replacement of an intervertebral disc, said endoprosthesis comprising a filler retention membrane, said filler retention membrane comprising an interior, a neck and an orifice, wherein said neck and said orifice are disposed substantially within the interior of the filler retention membrane.

7. The endoprosthesis of claim 6, wherein said orifice comprises one or more sealed regions.

8. The endoprosthesis of claim 7, wherein said one or more sealed regions is disposed laterally with respect to said orifice.

9. The endoprosthesis of claim 8, wherein said one or more sealed regions is disposed contiguously to said orifice.

10. The endoprosthesis of claim 7, wherein said neck comprises one or more exterior walls, and wherein said one or more sealed regions is disposed contiguously to said one or more exterior walls.

11. The endoprosthesis of claim 6, wherein said neck is anchored within the interior of said filler retention membrane.

12. The endoprosthesis of claim 6 wherein said neck comprises an interior, and wherein said interior comprises a substantially solid material.

13. An endoprosthesis for partial or complete replacement of an intervertebral disc, said endoprosthesis comprising an exterior and an interior, an exterior port and an interior port and a passage therebetween, wherein said exterior port is offset with respect to said interior port.

14. The endoprosthesis of claim 13, wherein said passage comprises a non-linear configuration.

15. The endoprosthesis of claim 14, wherein said passage comprises an ‘S’ shaped configuration.

16. The endoprosthesis of claim 14, wherein said passage comprises a ‘Z’ shaped configuration.

17. The endoprosthesis of claim 13, wherein said passage comprises a substantially solid material.

18. An endoprosthesis for partial or complete replacement of an intervertebral disc, said endoprosthesis comprising a principle nuclear region and a reinforcement region, wherein said reinforcement region comprises a cross-sectional configuration, wherein said cross-sectional configuration comprises a polygonal configuration.

19. The endoprosthesis of claim 18, wherein said principle nuclear region comprises an interior and an exterior, and wherein said reinforcement region is disposed substantially within the interior of said principle nuclear portion.

20. The endoprosthesis of claim 18, wherein said principle nuclear region comprises an interior and an exterior, and wherein said reinforcement region is disposed substantially about the exterior of the said principle nuclear region.

21. An endoprosthesis for partial or complete replacement of an intervertebral disc, said endoprosthesis comprising a primary filler retention membrane comprising an interior, and one or more secondary filler retention membranes disposed within said primary filler retention membrane.

22. The endoprosthesis of claim 21 wherein said primary and said secondary filler retention membranes comprise a delivery configuration and a deployed configuration.

23. The endoprosthesis of claim 21 further comprising one or more means for measuring pressure within the interior of at least one of said primary or secondary filler retention membranes.
